(19) United States
(12) Patent Application Publication
(10) Pub. No.: US 2023/0111504 A1
(43) Pub. Date: Apr. 13, 2023

(54) CORONAVIRIDAE INFECTION AND AVERMECTINS

(71) Applicant: Huvepharma EOOD, Sofia (BG)

(72) Inventors: Kiril Petrov Domuschiev, Sofia (BG); Georgi Petrov Domuschiev, Sofia (BG)

(73) Assignee: Huvepharma EOOD, Sofia (BG)

(21) Appl. No.: 17/593,265

(22) PCT Filed: May 21, 2021
(86) PCT No.: PCT/EP2021/063721

§ 371 Date: Sep. 14, 2021

(30) Foreign Application Priority Data
Jun. 19, 2020 (GB) 2009387.8
Dec. 24, 2020 (GB) 2020611.6
Feb. 5, 2021 (GB) 2101630.8

(51) Int. Cl. A61K 31/7048; A61P 31/14

(52) U.S. Cl. CPC A61K 31/7048; A61P 31/14

(57) ABSTRACT

The present invention relates to new uses and methods of treatment. Specifically, the invention relates to new uses of Avermectins such as ivermectin for the treatment of Coronaviridae infection such as MERS, SARS or COVID-19 infection. In particular, the invention relates to new uses of Avermectins such as ivermectin for the treatment of COVID-19 infection.

The present invention relates to new uses and methods of treatment. Specifically, the invention relates to new uses of Avermectins such as ivermectin for the treatment of Coronaviridae infection such as MERS, SARS or COVID-19 infection. In particular, the invention relates to new uses of Avermectins such as ivermectin for the treatment COVID-19 infection.

### BACKGROUND

The Coronaviridae family of viruses or ‘coronaviruses’, are a large, enveloped, plus (+) strand RNA genome viruses that cause highly prevalent diseases in vertebrate animals and can cause illnesses with varying severity. Coronaviruses have the largest genomes of all RNA viruses and replicate by a unique mechanism which results in high frequency of recombination. Most coronaviruses naturally infect only one species or several closely related species, with virus replication often limited to epithelial cells of the respiratory or enteric tracts and macrophages. Isolation of coronaviruses from infected individuals if often difficult and sometimes requires differentiated cells from natural host species.

In humans, coronaviruses can cause illnesses ranging from common cold to respiratory symptoms, fever or chill, continuous cough, shortness of breath, muscle and body aches, breathing difficulties and new loss of taste or smell. In healthy individuals and more commonly in those with underlying health conditions, coronaviruses infection can cause acute shortness of breath, pneumonia, severe acute respiratory syndrome, lung failure, kidney failure and often death. Seroepidemiological surveys and virus-isolation studies indicate that coronaviruses cause about 15-20% of upper respiratory tract infections in humans and suggest a possible role in other human diseases such as pneumonia and myocarditis.

Two human coronaviruses, Middle East respiratory syndrome (MERS) and severe acute respiratory syndrome (SARS), have been known to frequently cause severe symptoms. Very recently, a new rapidly spreading coronavirus strain that has not previously been identified in humans, the 2019 novel coronavirus (2019-nCoV), has been reported. The 2019-nCoV is a Betacoronavirus, like MERS and SARS, all of which have their origins in bats.

Coronavirus disease 2019 (COVID-19) was discovered in Hubei Province, China in December 2019. A cluster of patients were admitted with fever or chill, cough, shortness of breath, and other symptoms such as nausea or vomiting, diarrhea, sore throat or headache. The World Health Organization (WHO) declared severe acute respiratory syndrome coronavirus 2 (SARS-CoV-2) infection (causing coronavirus disease 2019, COVID-19) a pandemic disease on Mar. 11, 2020 (Sun et al., 2020).

Coronavirus disease 2019 (COVID-19) has become a public health emergency.

The novel coronavirus was named SARS-CoV-2, the pathogen causing COVID-19. The causative agent of the current COVID-19 pandemic, SARS-CoV-2, is a single stranded positive sense RNA virus that is closely related to severe acute respiratory syndrome coronavirus (SARS-CoV). Studies on SARS-CoV proteins have revealed a potential role for IMPα/β1 during infection in signal-dependent nucleocytoplasmic shutting of the SARS-CoV Nucleocapsid protein. In addition, the SARS-CoV accessory protein ORF6 has been shown to antagonize the antiviral activity of the STAT1 transcription factor by sequestering IMPα/β1 on the rough ER/Golgi membrane.

There is a simple but critical number which help our understanding the threat a particular contagious disease poses to the population. One reliable method of rating a disease's ability to spread from subject to subject is based on the reproduction number. Asymptomatic individuals are as infectious as symptomatic individuals and are therefore capable of further spreading the disease. SARS-CoV-2 can be transmitted from human to human and is suggested to have a zoonotic origin.

It is estimated that a SARS-CoV-2-infected person will infect approximately three new people (the reproductive value (R value) is averaged to be ˜3.28). The symptoms can vary, with some patients remaining asymptomatic, while others present with high temperature, new continuous cough, fatigue, loss or change to sense of smell or taste and a host of other symptoms. The symptoms may be similar to patients with influenza or the common cold. At this stage, the most likely mode of transmission is thought to be through direct subject to subject contact and droplet spread.

Main clinical symptoms include fever, continuous cough, myalgia or fatigue, expectoration, sore throat, conjunctivitis and dyspnea plus a number of less manifested and recently reported clinical symptoms of almost all body systems and organs, including but limited to encephalitis, Meniere-like syndrome, gastrointestinal, cardio-vascular, renal and skin manifestations correlating with changes in a number of paraclinical and laboratory parameters. While most patients do not experience severe symptoms, and some remain asymptomatic or oligosymptomatic, one meta-analysis found that approximately 14-18% of cases were representing a severe clinical picture (Crump A., 2017). Fatality rates are estimated to be approximately 4-7% at this time (Siddiqi and Mehra, 2020; Crump A., 2017).

At present, 3-stage classification of the disease has been described (Gloeckner et al., 2010). The initial stage occurs at the time of inoculation and early establishment of the disease. For most people, this involves an incubation period associated with mild and often non-specific symptoms such as malaise, mild fever, sore throat and a continuous cough e.g. dry cough. Treatment at this stage is primarily targeted towards symptomatic relief. Should a viable anti-viral therapy be proven beneficial, the target pool of infected patients during this particular stage of the disease may reduce duration of symptoms, minimize transmission and reduce severity of disease. It has been suggested that intervention or treatment at this stage of disease may present a powerful tool in limiting COVID-19 life-threatening and incapacitating capabilities.

In the second stage of established pulmonary disease, viral multiplication and localised inflammation associated with rapid influx of neutrophils and other innate immune system cells in the lungs is a common place. During this stage, patients develop an interstitial lung disease (viral pneumonia), with cough such as dry cough, fever and possibly hypoxia. Treatment would primarily consist of supportive measures and available more aggressive (and expensive) anti-viral therapies which are mainly administered via intravenous route of application. If hypoxia is not reverted via these additional treatments (Stage IIb), it is likely that patients w % ill progress to requiring mechanical ventilation and, in that situation, the use of anti-inflammatory therapy may be useful.

A minority of COVID-19 patients will undergo transition into the third and most severe stage of illness, which manifests as an extra-pulmonary systemic hyperinflammation syndrome. In this stage, markers of systemic inflammation appear to be elevated, including cytokines and interleukin (IL). In this stage, the patients experience shock, vasoplegia, respiratory failure and even cardiopulmonary collapse. Systemic organ involvement, even myocarditis, would manifest during this stage. Tailored therapy in Stage III relies on the use of immunomodulatory agents, to reduce systemic inflammation before it overwhelmingly results in multi-organ dysfunction.

According to GlobalData 4 Feb. 2020, Clinical Trials Database: 69.2%, were investigating treatments for general Coronaviridae or Betacoronavirus infections, but the other 30.8% were investigating specific therapies for MERS, SARS, or 2019-nCoV. A total of 13.3% of trials are indicated for MERS, with 50% in Phase I development, 43.8% currently ongoing, and 18.8% planned. There have been no cases of SARS reported worldwide since 2004, so the 7.5% of trials with this indication are either completed or withdrawn.

Currently, 10% of these clinical trials are in response to the new 2019-nCoV outbreak. Of these, 66.7% are planned to be initiated and the other 33.3% are already recruiting. At 41.7%, the majority are in Phase IV, followed by 33.3% in Phase 0 and 25% in Phase III. All sponsors of these 2019-nCoV trials are institutions located in China, besides one, which is a company-sponsored trial by Gilead Sciences. The Gilead trial is also taking place in China, specifically in Wuhan, and is a Phase III trial for patients with mild to moderate pneumonia caused by 2019-nCoV. The intervention is remdesivir (GS-5734), an experimental treatment for the Ebola virus infection, SARS, MERS, and nipah and zika virus infections. The drug candidate is a small molecule prodrug of adenine nucleotide analogue and acts by blocking the viral RNA replication process. The number of clinical trials indicated for 2019-nCoV is expected to rise as this outbreak continues.

Our understanding of the clinical spectrum of 2019-nCoV infection is limited. Complications such as severe pneumonia, respiratory failure, gastric inflammation, acute respiratory distress syndrome (ARDS) and cardiac injury, including fatal outcomes, have been reported on global scale. Also, little if anything is known about the long-term impact on patients after COVID-19 infection. Since the majority of reported cases were identified on the basis of their pneumonia diagnosis and this may bias reporting toward more severe outcomes, both treatment and description of the medical condition and progression of infection represent an acutely unmet challenge. Despite the intensive efforts to find therapeutic antiviral agents to manage the emergencies, reports on specific and effective drugs or vaccines with low toxicity and desired efficiency are rare if not absent.

Thus, currently, there are no effective treatments against the new coronavirus SARS-CoV-2 or COVID-19. There are also no treatments which can be widespread and accepted as treatment standards.

Therefore, there is an urgent need for developing not only effective treatments against SARS-CoV-2 or COVID-19 but also treatment which can be widely adopted and lack toxicity.

### SUMMARY

The present invention relates to new uses and treatment methods that alleviate, abrogate, or otherwise reduce or stop any one or more of the above clinical symptoms by administering an active ingredient or substance.

Specifically, the invention relates to new uses of clinically safe Avermectins, for the treatment of Coronaviridae infected subjects.

In one aspect, there is provided a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for use in the treatment of a subject infected with a Coronaviridea family member.

In some embodiments, there is provided a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for use in the treatment of a subject infected with a Coronaviridea family member selected from the group consisting of MERS, SARS or COVID-19.

In some embodiments, there is provided a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for use in the treatment of a subject infected with COVID-19.

In some embodiments, the subject is administered with a therapeutically effective amount of the substance in a free form or in the form of a physiologically acceptable derivative and/or salt thereof.

In some embodiments, the therapeutically effective amount is achieved by a regimen of administration of the substance in a free form or in the form of a physiologically acceptable derivative and/or salt thereof.

In some embodiments, the regimen of administration of the substance in a free form or in the form of a physiologically acceptable derivative and/or salt thereof, is carried out using methods known in the art.

In some embodiments, the regimen comprises one or more types of administration of the substance, in a free form or in the form of a physiologically acceptable derivative and/or salt thereof.

In some embodiments, the regimen of administration of the substance in a free form or in the form of a physiologically acceptable derivative and/or salt thereof, is suitable for peroral administration, pulmonary administration, intravenous administration or subcutaneous administration.

In some embodiments, the regimen of administration of the substance in a free form or in the form of a physiologically acceptable derivative and/or salt thereof, comprises one or more of peroral administration, pulmonary administration, intravenous administration and subcutaneous administration.

It will be appreciated by those skilled in the art that the amount or dose of the substance, such as ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13 (i.e. C13), in a free form or in the form of a physiologically acceptable derivative and/or salt thereof, as contemplated in the present invention, will vary with the nature or severity of the condition being treated, the type of the viral infection, the stage of the infection, the viral load to which the subject was exposed upon initial encounter with the virus, the age, weight and the overall condition of the subject, and will be ultimately at the discretion of the attendant physician. The amount or dose of the substance, is preferably pharmaceutically relevant for the intended use and desired outcome such as amelioration, prophylaxis, or reversal of a disease or disorder, or of at least one discernible symptom thereof caused by or associated with an infection by a Coronaviridea family member. The amount or dose of the substance, is preferably pharmaceutically relevant for the intended use and desired outcome such as amelioration, prophylaxis, or reversal of a disease or disorder, or of at least one discernible symptom thereof caused by or associated with an infection by a Coronaviridea family member selected from the group consisting of MERS, SARS or COVID-19. In some embodiments, the amount or dose of the substance, is preferably pharmaceutically relevant for the intended use and desired outcome such as amelioration, prophylaxis, or reversal of a disease or disorder, or of at least one discernible symptom thereof caused by or associated with COVID-19.

In some embodiments, the dose of the substance may conveniently be presented in a single dose or as divided doses administered at appropriate intervals, for example as two, three, four or more sub-doses per day or per administration.

In some embodiments, the substance is ivermectin in a free form or in the form of a physiologically acceptable derivative and/or salt thereof.

In some embodiments, the substance is ivermectin.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, leads to a clinical improvement in a subject infected with a Coronaviridea family member.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, leads to a clinical improvement in a subject infected with COVID-19.

In some embodiments, the clinical improvement is statistically significant compared to a placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, leads to a reduction in severity of disease in a subject infected with a Coronaviridea family member.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, leads to a reduction in severity of disease in a subject infected with COVID-19.

In some embodiments, the reduction in severity of disease is statistically significant compared to a placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, leads to a reduction of viral load in a subject infected with a Coronaviridea family member.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, leads to a reduction of viral load in a subject infected with COVID-19.

In some embodiments, the present treatment leads to an accelerated or faster reduction in the viral load in the treated patient compared to a placebo.

In some embodiments, the reduction of the viral load is statistically significant.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, leads to a reduction of one or more markers selected from the group of consisting of inflammatory markers, CBC markers and predictive markers, in a subject infected with COVID-19.

In some embodiments, the present treatment leads to reduction of one or more predictive markers.

In some embodiments, the present treatment leads to reduction of one or more predictive markers selected from the group consisting of lymphocyte count; C reactive protein; D-dimer; LDH; ALAT; and ASAT, in a subject infected with COVID-19.

In some embodiments, the present treatment leads to reduction of D-dimer marker levels in a subject infected with COVID-19.

In some embodiments, the reduction of the predictive marker in a treated patient is statistically significant compared to a placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in severity of disease in a subject infected with COVID-19, about 3, 4, 5 or 6 days after first dose of peroral administration of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in viral load in a subject infected with COVID-19, about 3, 4, 5 or 6 days after first dose of peroral administration of the substance compared to placebo.

In an aspect of the present invention there is provided a use of a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for preparing an antiviral medicament for the therapeutic treatment of a subject infected with a Coronaviridea family member.

In some embodiments, the present invention is directed a use of a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for preparing an antiviral medicament for the therapeutic treatment of a subject infected with a with a Coronaviridea family member selected from the group consisting of MERS, SARS or COVID-19.

In some embodiments, the present invention is directed to a use of a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for preparing an antiviral medicament for the therapeutic treatment of a subject infected with COVID-19.

In some embodiments, the use of the substance is ivermectin in a free form or in the form of a physiologically acceptable derivative and/or salt thereof.

In some embodiments, the use of the substance is ivermectin.

It will be appreciated by those skilled in the art that the amount or dose of the substance, such as, ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, as contemplated in the present invention, will vary with the nature or severity of the condition being treated, the type of the viral infection, the stage of the infection, the viral load to which the subject was exposed upon initial exposure to the virus, the age, weight and the overall condition of the subject, and will be ultimately at the discretion of the attendant physician. The amount or dose of the antiviral medicament, is preferably pharmaceutically relevant for the intended use and desired outcome such as amelioration, prophylaxis, or reversal of a disease or disorder, or of at least one discernible symptom thereof caused by or associated with an infection by a Coronaviridea family member selected from the group consisting of MERS, SARS or COVID-19. In some embodiments, the antiviral medicament is preferably pharmaceutically relevant for the intended use and desired outcome such as amelioration, prophylaxis, or reversal of a disease or disorder, or of at least one discernible symptom thereof caused by or associated with an infection by COVID-19.

In some embodiments, the dose of the substance, will typically be in the range of about 2 to about 2000 mg/ml per administration, dependent upon the route of administration.

In some embodiments, the dose of the substance may conveniently be presented in a single dose or as divided doses administered at appropriate intervals, for example as two, three, four or more sub-doses per day or per administration.

In some embodiments, the dose of substance per administration will typically be in the range of about 0.1 to 2000 mg/kg of body weight, about 0.15 to 1750 mg/kg of body weight, about 0.2 to 1700 mg/kg of body weight, about 0.3 to 1500 mg/kg of body weight, about 0.5 to 1250 mg/kg of body weight, about 1 to 1000 mg/kg of body weight, about 2 to 900 mg/kg of body weight, about 3 to 800 mg/kg of body weight, about 4 to 700 mg/kg of body weight, about 5 to 600 mg/kg of body weight, about 10 to 500 mg/kg of body weight, administered as one, two, three, four or more doses or sub-doses per day or per administration.

In some embodiments, the dose is 400 mg/kg of body weight administered as one, three, four or more doses or sub-doses per day or per administration.

In some embodiments, the substance is adapted for peroral administration. In some embodiment, where the substance is adapted for peroral administration, a daily dose will typically be within the range of about 10 to 1500 μg/kg of body weight, about 20 to 1250 μg/kg of body weight, about 30 to 1000 μg/kg of body weight, about 500 to 750 μg/kg of body weight. In some embodiments, the peroral administration daily dose is 400 μg/kg of body weight.

In an aspect of the present invention there is provided a method for treating a subject infected with a Coronaviridea family member, the method comprising administering a therapeutically effective amount of a medicament selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof.

In some embodiments, there is provided a method for treating a subject infected with a Coronaviridea family member selected from the group consisting of MERS, SARS or COVID-19, the method comprising administering a therapeutically effective amount of a medicament selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof.

In some embodiments, there is provided a method for treating a subject infected with COVID-19, method comprising administering a therapeutically effective amount of a medicament selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof.

In some embodiments, there is provided a method for treating a subject, wherein the medicament is ivermectin in a free form or in the form of a physiologically acceptable derivative and/or salt thereof.

In some embodiments, there is provided a method for treating a subject, wherein the medicament is ivermectin.

In an aspect of the present invention there is provided a composition for treatment, prevention or amelioration of Coronaviridea family member infection, comprising ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof.

In some embodiments, there is provided a composition for treatment, prevention or amelioration of Coronaviridea family member infection selected from the group consisting of MERS, SARS or COVID-19, comprising administering a therapeutically effective amount of a medicament selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof.

In some embodiments, there is provided a composition for treatment, prevention or amelioration of COVID-19 infection, comprising administering a therapeutically effective amount of a medicament selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof.

In some embodiments, there is provided a composition comprising ivermectin in a free form or in the form of a physiologically acceptable derivative and/or salt thereof.

In some embodiments, there is provided a composition comprising ivermectin.

In some embodiments, the treatment leads to amelioration, prophylaxis, or reversal of a disease or disorder, or of at least one discernible symptom thereof, caused by or associated with Coronaviridae infection such as MERS, SARS or COVID-19 infection.

In some embodiments, the treatment leads to amelioration, prophylaxis, or reversal of a disease or disorder, or of at least one discernible symptom thereof, caused by or associated with Coronaviridae infection such as MERS, SARS or COVID-19 infection, by at least 10%, at least 20%, at least 30%, at least 40%, at least 50%, at least 60%, at least 70%, at least 80%, at least 81%, 82%, 83%, 84%, 85%, 86%, 87%, 88%, 89%, 90%, 91%, 92%, 93%, 94%, 95%, 96%, 97%, 98%, 99% or 100%.

In some embodiments, the treatment leads to substantial cure of the disease or disorder caused by or associated with Coronaviridae infection such as MERS, SARS or COVID-19 infection.

### BRIEF DESCRIPTION OF THE DRAWINGS

FIG. 1. Graph showing COVID-19 clinical trial first interim stage criteria. The study begins, and response data is collected for subjects, moving toward the first-stage target number of subjects, until a decision to perform an analysis on the existing data is made.

FIG. 2. Graph showing COVID-19 clinical trial second stage criteria. The z-statistic is computed from the sample proportions of the complete data from each group. The z-statistic is compared to the boundary and a decision of efficacy or futility is made.

FIG. 3. Clinical improvement by visit day. Ivermectin gives better results at days 4 and 5, and after that the difference decreases and goes statistically insignificant.

FIG. 4. Odds Ratio over Visit Day. Odds ratio measurement over the visit day clearly demonstrated that the chances of clinical improvement for Ivermectin arm are more than twice higher than for SoC arm only at about the fourth day.

FIG. 5. Predictive marker C reactive protein by visit.

FIG. 6. Significant reduction of predictive marker D-dimer levels in treated subjects compared to standard of case control.

### DETAILED DESCRIPTION

Throughout this disclosure, various scientific publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings.

As used in the specification and claims, the singular form “a,” “an” and “the” include singular and plural references unless the context clearly dictates otherwise. For example, the term “Avermectin class” or “Avermectin” includes a single or plurality of avermectins or substances selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13 (C13), in a free form or in the form of a physiologically acceptable derivative and/or salt thereof.

As used herein the term “substance” refers to avermectin class or pharmaceutical agents or therapeutically active ingredients. In some embodiments the substance includes a single or plurality avermectins or substances selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13 (C13), in a free form or in the form of a physiologically acceptable derivative and/or salt thereof. In some embodiments the substance is ivermectin.

All numbers or numerals as used herein that indicate amounts, ratios of materials, physical properties of materials, and/or use are to be understood as modified or qualified by the term “about,” except as otherwise explicitly indicated.

As used herein, the term “about” includes the recited number or number and +/−10% from the recited numeral or number. By way of non-limiting example, the term “about ten (10)” would encompass nine (9) to eleven (11) or 9-11.

As used herein, the term “subject” means any animal, such as a vertebrate, preferably a mammal such as human, to whom will be or has been administered substances, compounds or compositions according to embodiments of the invention. Preferably, a subject is in need of, or has been the object of observation or experiment of, treatment or prevention of Coronaviridae infection. Preferably, a subject is in need of, or has been the object of observation or experiment of, treatment or prevention of Coronaviridae infection such as MERS, SARS or COVID-19 infection. Preferably, a subject is in need of, or has been the object of observation or experiment of, treatment or prevention of COVID-19 infection.

As used herein, the term “study population” or “S-population” is used interchangeably with the term “trial population” or “T-population” and refer to subjects that fulfill all eligibility criteria to be involved in the study to whom the study substance of the present invention is administered. In some embodiments the S-population comprises randomised subject to whom the study substance of the present invention is administered. In some embodiments the S-population represents all randomised subjects to whom the study substance of the present invention is administered. As used herein the term “randomised” refers to reducing or eliminating bias in the way the treatment is allocated for administration to a subject.

As used herein the term “eligibility criteria” refers to selection criteria for subjects such as subject inclusion criteria and subject exclusion criteria which are used to determine whether a subject is to be involved in the study or the trail to whom the study substance of the present invention is administered or not.

As used herein the term “symptom” refers to for example fever, new cough, myalgia or fatigue, expectoration, sore throat, conjunctivitis and dyspnea.

As used herein, the term “treatment” or “treating” refers to an amelioration, prophylaxis, or reversal of a disease or disorder, or of at least one discernible symptom thereof. It is also contemplated that the treatment, as described herein throughout and based on clinical trials data, leads to one or more of clinical improvement, reduction in the severity of disease and reduction in viral load in a patient. In some embodiments, “treatment” or “treating” refers to an amelioration, prophylaxis, or reversal of at least one measurable physical parameter related to the disease or disorder being treated, not necessarily discernible in or by the mammal. In some embodiments, “treatment” or “treating” refers to inhibiting or slowing the progression of a disease or disorder, either physically, e.g., stabilisation of a discernible symptom, physiologically, e.g., stabilisation of a physical parameter, or both. In some embodiments, “treatment” leads to partial or complete remission of the disease or disorder. In some embodiments, “treatment” leads to clinical improvement in a subject infected with Coronaviridae family member such as COVID-19. In some embodiments, “treatment” leads to reduction in the severity of disease in a subject infected with Coronaviridae family member such as COVID-19. In some embodiments, “treatment” leads to reduction in viral load in a patient infected with Coronaviridae family member such as COVID-19.

Without wishing to be bound by theory, elevated blood levels of different markers such as inflammatory markers, CBC markers and predictive markers have been associated with disease state such as for example viral infection. Thus, in some embodiments of the present invention, the predictive markers are selected from the group consisting of lymphocyte count; C reactive protein; D-dimer; LDH; ALAT; and ASAT. In some embodiments, the predictive marker is selected from C reactive protein or D-dimer. In some embodiments, the predictive marker is D-dimer. In some embodiments of the present invention, “treatment” leads to reduction of one or more predictive markers selected from the group consisting of lymphocyte count; C reactive protein; D-dimer; LDH; ALAT; and ASAT. In some embodiments of the present invention, “treatment” leads to reduction of a predictive marker selected from C reactive protein or D-dimer. In some embodiments of the present invention, “treatment” leads to reduction of the predictive marker D-dimer. In some embodiments of the present invention, “treatment” leads to statistically significant reduction of the predictive marker D-dimer.

The present invention relates to new uses and treatment methods that alleviate, abrogate, or otherwise reduce or cure any one or more symptoms caused by or associated with a Coronaviridae infection by administering or applying an active ingredient or substance. Specifically, the invention relates to new uses of Avermectins, for the treatment of Coronaviridae infected subjects. In some embodiments, the treatment leads to substantial cure of the disease or disorder caused by or associated with Coronaviridae infection such as MERS, SARS or COVID-19 infection.

While the emphasis of the present disclosure resides with subjects, those of skill in the art will readily recognise that the present invention is also equally applicable and effective to non-human subjects (i.e. vertebrate animals) such as, for example, livestock (e.g. cattle, horses and sheep), exotic animals (e.g. pandas, big cats such as tigers, lions and pumas, elephants, bats and similar animals) and also companion animals (such as dogs and cats), particularly where a disease or condition is associated with or caused by Coronaviridae infection such as MERS, SARS or COVID-19 infection.

The present inventors surprisingly discovered that administration of a substance selected from the Avermectin group of substances would treat a disease or condition associated with or caused by Coronaviridae infection such as MERS, SARS or COVID-19 infection. The present inventors also observed that administration of a substance selected from the Avermectin group of substances would lead to amelioration, prophylaxis, or reversal of a disease or disorder, or of at least one discernible symptom thereof, caused by or associated with Coronaviridae infection such as MERS, SARS or COVID-19 infection.

It would be known by those of skill in the art that the Avermectin group of substances comprises ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof.

In one aspect, there is provided a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for use in the treatment of a subject infected with a Coronaviridea family member.

Ivermectin is a member of the Avermectin class, which has been shown in immunopharmacological studies to exert anti-inflammatory effects by inhibiting lipopolysaccharide-induced production of inflammatory cytokines, such as tumour necrosis factor alpha and interleukin (IL)-1b, while upregulating the anti-inflammatory cytokine IL-10. It is a semi-synthetic derivative isolated from the fermentation of Streptomyces avermitilis, that belongs to the avermectin family of macrocyclic lactones. Ivermectin is a mixture containing 5-O-demethyl-22,23-dihydroavermectin Ala plus 5-O-demethyl-25-de(1-methylpropyl)-25-(1-methylethyl)-22,23-dihydroavermectin Ala, generally referred to as 22,23-dihydroavermectin B1a and B1b or H2B1a and H2B1b, respectively. The respective empirical formulas of H2B1a and H2B1b are C48H74014 and C47H72014 have molecular weights of 875.10 and 861.07 respectively.

Ivermectin is a macrocyclic lactone derivative, its therapeutic effect is thought to be prominently due to its anti-inflammatory properties, similar to that of other macrolides. Avermectin has been reported to exert anti-inflammatory effects by inhibiting lipopolysaccharide-induced production of inflammatory cytokines. In addition to its anti-inflammatory mode of action, ivermectin possesses antiparasitic properties. Its predecessor, avermectin, is an antiparasitic agent of agricultural importance first isolated in 1974. Several studies support ivermectin's role in the effective oral treatment of cutaneous demodicidosis (in combination with topical permethrin cream) and scabies, as well as topical treatment of head lice. Ivermectin causes death of parasites, primarily through binding selectively and with high affinity to glutamate-gated chloride channels, which occur in invertebrate nerve and muscle cells. This leads to the interruption of nerve impulses, causing paralysis and death of parasitic organisms. Ivermectin is known to act on Demodex mites in localized and generalized demodicidosis in animals and in humans.

In the present invention, studies were conducted to evaluate the efficacy and safety of a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, in treating COVID-19 infection. In some embodiments the studies were conducted to evaluate the efficacy and safety of ivermectin in treating COVID-19 infection.

As used in the present invention, a therapeutically effective amount of substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, can be delivered to a patient as part of a regimen.

As used herein the term “regimen” refers to a plan or a set of rules of different possible routs or modes of administration, preferably to achieve a therapeutically effective amount of substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, in a subject.

It would be known to those of skill in the art that different regimens may be employed in the context of the present invention.

In some embodiments, the regimen of administration of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is carried out using methods known in the art.

As used herein the term “administration” should be understood to encompass for example peroral, intravenous, parenteral, inhalation, pulmonary, rectal, nasal, topical (e.g., transdermal and intraocular), intravesical, intrathecal, enteral, pulmonary, intralymphatic, intracavital, vaginal, transurethral, intradermal, aural, intramammary, buccal, orthotopic, intratracheal, intralesional, pereutaneous, endoscopical, transmucosal, sublingual, intestinal administration and combinations thereof.

Peroral Administration: The substance of the invention can be formulated to take the form of tablets or capsules prepared by conventional means with one or more pharmaceutically acceptable carriers (e.g., excipients such as binding agents, fillers, lubricants and disintegrants).

Parenteral Administration: The substance of the present invention can be formulated for parenteral administration by injection (e.g., by bolus injection or continuous infusion). Formulations for injection can be presented in unit dosage form in ampoules or in multi-dose containers with an optional preservative added. The parenteral preparation can be enclosed in ampoules, disposable syringes or multiple dose vials made of glass, plastic or the like. The formulation can take such forms as suspensions, solutions or emulsions in oily or aqueous vehicles, and can contain agents such as suspending, stabilizing and/or dispersing agents. For example, a parenteral preparation can be a sterile injectable solution or suspension in a nontoxic parenterally acceptable diluent or solvent (e.g., as substance in 1,3-butanediol solution). Some of the acceptable vehicles and solvents that can be employed include for example water, Ringer's solution, and isotonic sodium chloride solution. In addition, sterile, fixed oils are conventionally employed as a solvent or suspending medium. In addition, fatty acids such as oleic acid can be used in the parenteral preparation.

Controlled-Release Administration: Controlled-release (or sustained-release) preparations can be formulated to extend the activity of a substance and reduce dosage frequency. Controlled-release preparations can also be used to affect the time of onset of action or other characteristics, such as blood levels of the substance, and consequently affect the occurrence of any side effects.

Controlled-release preparations can be designed to initially release an amount of a substance that produces the desired therapeutic effect, and gradually and continually release other amounts of the substance to maintain the level of therapeutic effect over an extended period of time. In order to maintain a near-constant level of a substance in the body, the substance can be released from the dosage form at a rate that will replace the amount of substance being metabolised and/or excreted from the body. The controlled-release of a substance can be stimulated by various inducers, e.g., change in pH, change in temperature, enzymes, water, and/or other physiological conditions or molecules.

Controlled-release systems can include, for example, an infusion pump which can be used to administer the substance in a manner similar to that used for delivering insulin or chemotherapy to the body generally, or to specific organs or tumours. Typically, using such a system, the substance is administered in combination with a biodegradable, biocompatible polymeric implant that releases the substance over a controlled period of time at a selected site. Example polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage. Examples include micro-dosing directly into the upper respiratory canals or distal alveoli in the lungs using suitably adapted bronchoscopy such as robotic assisted-bronchoscopy. Suitable bronchoscopes are available from Ambu, J&J-Monarch Platform and ProtheaX Inc.

Substances of the invention can be administered by other controlled-release means or delivery devices that are well known to those of skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions. Other methods of controlled-release delivery of substances of the invention will be known to the skilled person and are within the scope of the invention.

Inhalation Administration: Substances of the invention can be administered directly to the lung of a patient/subject by inhalation. For administration by inhalation, a substance can be conveniently delivered to the lung by a number of different devices. For example, a Metered Dose Inhaler (“MDI”) which utilises canisters that contain a suitable low boiling point propellant, e.g., dichlorodifluoromethane, trichlorofluoromethane, dichlorotetrafluoroethane, carbon dioxide or other suitable gas can be used to deliver a substance directly to the lung. MDI devices are available from a number of suppliers such as Aventis, Boehringer Ingleheim, GlaxoSmithKline, Merck & Co. and Vectura.

Alternatively, Dry Powder Inhaler (DPI) device can be used to administer a substance to the lung. DPI devices typically use a mechanism such as a burst of gas to create a cloud of dry powder inside a container, which can then be inhaled by the patient. DPI devices are also well known in the art and can be purchased from a number of vendors which include, for example, GlaxoSmithKline. Nektar Therapeutics, Innovata and Vectura. A popular variation is the multiple dose DPI (“MDDPI”) system, which allows for the delivery of more than a single dose. MDDPI devices are available from companies such as AstraZeneca, GlaxoSmithKline, Merck & Co. and Vectura. For example, capsules and cartridges of gelatin for use in an inhaler or insufflator can be formulated containing a powder mix of the substance of the invention and a suitable powder base such as lactose or starch for these systems. Examples of administration in the upper respiratory canals or distal alveoli in the lungs include using bronchoscopy such as robotic assisted-bronchoscopy. Suitable bronchoscopes are available from Ambu, J&J-Monarch Platform and ProtheaX Inc.

In some embodiments, the regimen of administration of the substance in a free form or in the form of a physiologically acceptable derivative and/or salt thereof, is suitable for peroral administration, pulmonary administration, intravenous administration or subcutaneous administration.

In some embodiments, the regimen of administration of the substance in a free form or in the form of a physiologically acceptable derivative and/or salt thereof, comprises one or more of peroral administration, pulmonary administration, intravenous administration and subcutaneous administration.

It will be appreciated by those skilled in the art that the amount or dose of the substance, such as ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, as contemplated in the present invention, will vary with the nature or severity of the condition being treated, the type of the viral infection, the stage of the infection, the viral load to which the subject was exposed upon initial encounter with the virus, the age, weight and the overall condition of the subject, and will be ultimately at the discretion of the attendant physician. The amount or dose of the substance, is preferably pharmaceutically relevant for the intended use and desired outcome such as amelioration, prophylaxis, or reversal of a disease or disorder, or of at least one discernible symptom thereof caused by or associated with an infection by a Coronaviridea family member. The amount or dose of the substance, is preferably pharmaceutically relevant for the intended use and desired outcome such as amelioration, prophylaxis, or reversal of a disease or disorder, or of at least one discernible symptom thereof caused by or associated with an infection by a Coronaviridea family member selected from the group consisting of MERS, SARS or COVID-19.

In some embodiments, the dose of the substance may conveniently be presented in a single dose or as divided doses administered at appropriate intervals, for example as two, three, four or more sub-doses per day or per administration.

In some embodiments, the substance is ivermectin in a free form or in the form of a physiologically acceptable derivative and/or salt thereof.

In some embodiments, the substance is ivermectin.

It will be appreciated by those skilled in the art that the amount of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, required in the present methods will vary with the nature or severity of the condition being treated, the age, weight, pre-existing medical treatments, and the overall condition of the subject, and will be ultimately at the discretion of the attendant physician.

In an aspect of the present invention there is provided a use of a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for preparing an antiviral medicament for the therapeutic treatment of a subject infected with a Coronaviridea family member. In some embodiments, the therapeutic treatment of the subject leads to significant reduction of symptoms such as fever or chill, continuous cough, shortness of breath, muscle and body aches, breathing difficulties and loss of taste or smell. In some embodiments, the therapeutic treatment of the subject leads to complete removal of symptoms such as fever or chill, continuous cough, shortness of breath, muscle and body aches, breathing difficulties and loss of taste or smell.

In some embodiments, the present invention is directed a use of a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for preparing an antiviral medicament for the therapeutic treatment of a subject infected with a with a Coronaviridea family member selected from the group consisting of MERS, SARS or COVID-19.

In some embodiments, the present invention is directed to a use of a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for preparing an antiviral medicament for the therapeutic treatment of a subject infected with COVID-19.

In some embodiments, the substance is ivermectin in a free form or in the form of a physiologically acceptable derivative and/or salt thereof.

In some embodiments, the substance is ivermectin.

It will be appreciated by those skilled in the art that the amount or dose of the substance, such as, ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, as contemplated in the present invention, will vary with the nature or severity of the condition being treated, the type of the viral infection, the stage of the infection, the viral load to which the subject was exposed upon initial exposure to the virus, the age, weight and the overall condition of the subject, and will be ultimately at the discretion of the attendant physician. The amount or dose of the antiviral medicament, is preferably pharmaceutically relevant for the intended use and desired outcome such as amelioration, prophylaxis, or reversal of a disease or disorder, or of at least one discernible symptom thereof caused by or associated with an infection by a Coronaviridea family member selected from the group consisting of MERS, SARS or COVID-19.

In some embodiments, the dose of the substance, will typically be in the range of about 2 to about 2000 mg/ml per administration, dependent upon the route of administration.

In some embodiments, the dose of the substance may conveniently be presented in a single dose or as divided doses administered at appropriate intervals, for example as two, three, four or more sub-doses per day or per administration. The desired dose may conveniently be presented in a single dose or as divided doses administered at appropriate intervals, for example as two, three, four or more sub-doses per day or per administration.

In some embodiments, the dose of the substance per administration will typically be in the range of about 0.1 to 2000 mg/kg of body weight, about 0.15 to 1750 mg/kg of body weight, about 0.2 to 1700 mg/kg of body weight, about 0.3 to 1500 mg/kg of body weight, about 0.5 to 1250 mg/kg of body weight, about 1 to 1000 mg/kg of body weight, about 2 to 900 mg/kg of body weight, about 3 to 800 mg/kg of body weight, about 4 to 700 mg/kg of body weight, about 5 to 600 mg/kg of body weight, about 10 to 500 mg/kg of body weight, administered as one, two, three, four or more doses or sub-doses per day or per administration. In some embodiments, the dose is 400 mg/kg of body weight administered as one, two, three, four or more doses or sub-doses per day or per administration.

Avermectin class of substances such as ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, which are suitable for administration according to the present invention such as injectable use or nebuliser use, include but is not limited to, sterile aqueous solutions (where water soluble) or dispersions and sterile powders for the extemporaneous preparation of sterile injectable or nebulisable solutions or dispersion. In all cases the form must be sterile and must be fluid. It must be stable under the conditions of manufacture and storage and must be preserved against the contaminating action of microorganisms such as bacteria and fungi. The carrier can be a solvent or dispersion medium containing, for example, sterile water, ethanol, polyol (for example, glycerol, propylene glycol and liquid polyethylene glycol and the like), suitable mixtures thereof, and vegetable oils. The proper fluidity can be maintained, for example, by the use of a coating such as lecithin, by the maintenance of the required particle size in the case of a dispersion, and by the use of surfactants. The preventions of the action of microorganisms can be brought about by various antibacterial and antifungal agents; for example, parabens, chlorobutanol, phenol, sorbic acid and thimerosal. In many cases, it will be preferable to include isotonic agents, for example, sugars or sodium chloride. Prolonged or delayed release of the injectable compositions can be brought about by the use of agents delaying release, for example, aluminum monostearate and gelatin.

Sterile injectable solutions or dispersions are generally prepared by incorporating the substance or active ingredient in the required amount in the appropriate solvent with various of the other ingredients enumerated above, as required, followed by filtered sterilization. Dispersions are also prepared by incorporating the various sterilized active ingredient into a sterile vehicle which contains the basic dispersion medium and the required other ingredients from those enumerated above. In the case of sterile powders for the preparation of sterile solutions, the preferred methods of preparation are vacuum drying and the freeze-drying technique, which yield a powder of the active ingredient or substance plus any additional desired ingredient from previously sterile-filtered solution thereof.

In some embodiments, the substance is adapted for peroral administration. As used herein the term “peroral” means being adapted or suitable for administration through or by way of the mouth.

In some embodiment, where the substance is adapted for peroral administration, a daily dose will typically be within the range of about 10 to 1500 μg/kg of body weight.

In some embodiment, where the substance is adapted for peroral administration, a daily dose will typically be within the range of about 20 to 1250 μg/kg of body weight.

In some embodiment, where the substance is adapted for peroral administration, a daily dose will typically be within the range of about 30 to 1000 μg/kg of body weight.

In some embodiment, where the substance is adapted for peroral administration, a daily dose will typically be within the range of about 500 to 750 μg/kg of body weight.

In some embodiments, the peroral administration daily dose is 400 μg/kg of body weight.

The inventors found that the substance may be conveniently administered to a subject by the peroral route, particularly in the form of a tablet or capsule (e.g. a tablet). In some embodiments, the particular dosage regimes contemplated in the invention are particularly suited to oral administration in the form of a tablet or capsule that is formulated such that the release of compounds used in the invention e.g. Avermectin class of substances such as one or more selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, from said tablet or capsule after oral administration is modified. The term “modified” or “modified release” as used herein in relation to the substance according to the invention or a used in any other context means release, which is not immediate release and is taken to encompass controlled release, sustained release, prolonged release, timed release, retarded release, extended release and delayed release. In some embodiments, the modified release substance is ivermectin.

The term “modified release dosage form” as used herein can be described as dosage forms whose drug-release characteristics of time course and/or location are chosen to accomplish therapeutic or convenience objectives not offered by conventional dosage forms such as a solution or an immediate release dosage form. Modified release solid oral dosage forms include both delayed and extended release substance or active ingredient. As used herein, references to Avermectin class of substances such as one or more selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, allowing for delayed or controlled released will be understood by those skilled in the art.

The term “immediate release” as used herein in according to the invention or used in any other context means release which is not modified release and releases more than 70% of the active ingredient within 60 minutes. In some embodiments, the immediate release substance is ivermectin.

The term “immediate release dosage form” as used herein can be described as dosage form which allows the drug to dissolve in the gastrointestinal contents, with no intention of delaying or prolonging the dissolution or absorption of the substance or active ingredient.

In an aspect of the present invention there is provided a method for treating a subject infected with a Coronaviridea family member, the method comprising administering a therapeutically effective amount of a medicament selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof.

In some embodiments, there is provided a method for treating a subject infected with a Coronaviridea family member selected from the group consisting of MERS, SARS or COVID-19, the method comprising administering a therapeutically effective amount of a medicament selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof.

In some embodiments, there is provided a method for treating a subject infected with COVID-19, method comprising administering a therapeutically effective amount of a medicament selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof.

In some embodiments, there is provided a method for treating a subject, wherein the medicament is ivermectin in a free form or in the form of a physiologically acceptable derivative and/or salt thereof.

In some embodiments, there is provided a method for treating a subject, wherein the medicament is ivermectin.

In an aspect of the present invention there is provided a composition for treatment, prevention or amelioration of Coronaviridea family member infection, comprising ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof.

In some embodiments, there is provided a composition for treatment, prevention or amelioration of Coronaviridea family member infection selected from the group consisting of MERS, SARS or COVID-19, comprising administering a therapeutically effective amount of a medicament selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof.

In some embodiments, there is provided a composition for treatment, prevention or amelioration of COVID-19 infection, comprising administering a therapeutically effective amount of a medicament selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof.

In some embodiments, there is provided a composition comprising ivermectin in a free form or in the form of a physiologically acceptable derivative and/or salt thereof.

In some embodiments, there is provided a composition comprising ivermectin.

In some embodiments, the treatment leads to amelioration, prophylaxis, or reversal of a disease or disorder, or of at least one discernible symptom thereof, caused by or associated with Coronaviridae infection such as MERS, SARS or COVID-19 infection.

In some embodiments, the treatment leads to amelioration, prophylaxis, or reversal of a disease or disorder, or of at least one discernible symptom thereof, caused by or associated with Coronaviridae infection such as MERS, SARS or COVID-19 infection, by at least 10%, at least 20%, at least 30%, at least 40%, at least 50%, at least 60%, at least 70%, at least 80%, at least 81%, 82%, 83%, 84%, 85%, 86%, 87%, 88%, 89%, 90%, 91%, 92%, 93%, 94%, 95%, 96%, 97%, 98%, 99% or 100%.

In some embodiments, the treatment leads to remission of the disease or disorder caused by or associated with Coronaviridae infection such as MERS, SARS or COVID-19 infection. In some embodiments, the treatment leads to complete remission of the disease or disorder caused by or associated with Coronaviridae infection such as MERS, SARS or COVID-19 infection.

As used herein the term “remission” refers to a partial or complete disappearance, cessation or bringing to an end, of manifestation of symptoms of a disease or disorder cause by or associated with Coronaviridae infection such as MERS, SARS or COVID-19 infection.

In some embodiments, remission refers to partial or complete disappearance, cessation or bringing to an end, of manifestation of symptoms of a disease or disorder cause by or associated COVID-19.

As used herein, the term “complete remission” is used interchangeably with the term “full remission” refers to a total disappearance of manifestations of symptoms of a disease or disorder caused by or associated with Coronaviridae infection such as MERS, SARS or COVID-19 infection.

In some embodiments the complete remission refers to complete disappearance of manifestation of symptoms of a disease of disorder cause by or associated COVID-19.

In some embodiments, a subject treated with a substance or composition of the present invention, is no longer a carrier of COVID-19. In some embodiments, a subject treated with a substance or composition according to the present invention is not able to infect other subjects. In some embodiments, the treatment of the resent invention is able to control the R value.

The term “regression” may be used interchangeably with the terms, “reduction”, “decrease” or “reduce” a disease or disorder, viral load, or of at least one discernible symptom or marker caused by or associated with Coronaviridae infection such as MERS, SARS or COVID-19 infection. In some embodiments, the reduction is at least 5%, 10%, 15%, 20%, 25%, 30%, 35%, 40%, 45%, 50%, 55%, 60%, 65%, 70%, 75%, 80%, 85%, 90%, 95% or 100% or greater compared to control or placebo. In some embodiments the reduction is at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to control or placebo. In some embodiments the regression or reduction is statistically significant. In some embodiments the effectiveness or the treatment of the present invention is assessed by a quantifying step. In some embodiments, regression is assessed by a quantifying step. In further embodiments, the quantifying step is performed on a sample. In further embodiments, the quantifying step is performed by an immunoassay. In some embodiments, the sample is one of a plasma sample, blood sample, sputum sample, lavage, synovial fluid, or combinations thereof.

In some embodiments, regression refers to a disease or disorder, or at least one discernible symptom caused by or associated with COVID-19. In some embodiments, regression is used to provide an indication of the extent by which, for example, disease, disorder or symptom is altered in terms of, for example, frequency of occurrence, intensity and severity. In some embodiments, regression means make less, bring down, lower or lessen, for example, fever in the treated subject. In some embodiments, reduction means make less, bring down, lower or lessen, the viral load in the treated patient. In some embodiments, the present treatment leads to an accelerated or faster reduction in the viral load in the treated patient compared to a placebo. In some embodiments, the reduction of the viral load is statistically significant.

As used herein, the term “therapeutically effective amount” will be understood to refer to plasma levels of the relevant substances such as one or more of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof, at which the relevant (i.e. normally associated) therapeutic effect of that substance will typically be observed. The term may refer to a range of plasma levels or to a specific plasma level. In some embodiments the therapeutically effective amount is that of ivermectin in a free form or in the form of a physiologically acceptable derivative and/or salt thereof. This amount will vary dependent upon a number of variables, including the physiological needs of the patient to be treated, the nature or severity of the condition being treated, the type of the viral infection, the stage of the infection, the viral load to which the subject was exposed upon initial encounter with the virus, the age, weight and the overall condition of the subject, and will be ultimately at the discretion of the attendant physician.

In some embodiments, reference to a therapeutically effective amount of a substance of the present invention per millilitre (/ml) will be understood to refer to an amount per millilitre of plasma (i.e. blood plasma of the subject). As used herein, the reference to molar concentration will be understood to refer to a concentration in plasma (i.e. blood plasma of the subject). In some embodiments the plasma concentration is that of ivermectin in a free form or in the form of a physiologically acceptable derivative and/or salt thereof.

In some embodiments, the subject has a plasma concentration of ivermectin, in a free form or in the form of a physiologically acceptable derivative and/or salt thereof, that is below about 50 to about 170 μg/ml (such as e.g. below about 50, about 70, about 90, about 110, about 130, about 150, or about 170 μg/ml).

In some embodiments, the subject has a plasma concentration of ivermectin, in a free form or in the form of a physiologically acceptable derivative and/or salt thereof, that is at least about 70 to about 700 μM (such as e.g. at least about 70, about 140, about 210, about 280, about 350, about 420, about 490, about 560, about 630 or about 700 μM).

In some embodiments, the subject has a plasma concentration of ivermectin, in a free form or in the form of a physiologically acceptable derivative and/or salt thereof, that is below about 350 to about 1200 μM (such as e.g. below about 350, about 490, about 630, about 770, about 910, about 1050, or about 1190 μM).

For the avoidance of doubt, the skilled person will understand that references to certain maximum amounts and concentrations in plasma may also require a minimum of a therapeutically effective amount in the plasma.

The skilled person will understand that references to certain maximum (i.e. where values are indicated as being “below”) and minimum (i.e. where values are indicated as being “at least”) amount and/or concentrations in plasma may be combined to form ranges (i.e. wherein the amount in plasma is in a range that is from the minimum value to the maximum value).

As used herein, the term “significant” when referring to for example reducing, enhancing, remission, amelioration, prophylaxis, or reversal, that is statistically significant, not due to chance alone, which has a p-value of 0.05 or less. In particular, the term “significant” can have a p-value of less than 0.05, 0.04, 0.03, 0.01, 0.005, 0.001, etc., when referring to for example reducing, enhancing, remission, amelioration, prophylaxis, or reversal of disease, disorder or symptom caused by or associated with Coronaviridae infection such as MERS, SARS or COVID-19 infection, for example when compared with the level or frequency of occurrence of infection in one or more non-treated patients or when compared to the level or frequency of occurrence of infection in the same patient observed at an earlier time point (e, g. comparison with a “base line” level or placebo). Those of skill in the relevant art would be familiar with different statistical calculation approaches, examples include, t-test, z-test, sample test, O'Brien-Fleming method for normally distributed data etc.

In some embodiments the significant reducing, enhancing, remission, amelioration, prophylaxis, or reversal, refers to statistically significant reduction, enhancement, remission, amelioration, prophylaxis, or reversal of disease, disorder or symptom caused by or associated with COVID-19.

It was surprisingly observed that a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, was able to lead to a statistically significant clinical improvement in a subject infected with a Coronaviridea family member.

It was surprisingly observed based that a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, was able to lead to a statistically significant reduction in severity of disease in a subject infected with a Coronaviridea family member.

It was surprisingly observed based on clinical trials data that a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, was able to lead to a statistically significant reduction of viral load in a subject infected with COVID-19.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant clinical improvement in a subject infected with COVID-19.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant clinical improvement in a subject infected with COVID-19, at least 3, 4, 5 or 6 days after first dose of peroral administration of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant clinical improvement in a subject infected with COVID-19, about 3 days after first dose of peroral administration of 400 μg/kg of body weight of daily dose of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant clinical improvement in a subject infected with COVID-19, about 4 days after first dose of peroral administration of 400 μg/kg of body weight of daily dose of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant clinical improvement in a subject infected with COVID-19, about 5 days after first dose of peroral administration of 400 μg/kg of body weight of daily dose of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant clinical improvement in a subject infected with COVID-19, about 6 days after first dose of peroral administration of 400 μg/kg of body weight of daily dose of the substance compared to placebo.

As used herein the term “clinical improvement” refers to WHO 9-category ordinal scale, developed to have a standardised measure of the COVID-19 disease severity. Clinical improvement is defined as a decrease on WHO ordinal scale score by at least one point.

It was surprisingly observed based on clinical trials data that a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, was able to lead to a clinical improvement, reduction in the severity of disease or reduction in viral load in a patient infected with COVID-19.

It was surprisingly and unexpectedly observed based on clinical trials data that a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, was able to lead to a statistically significant clinical improvement, reduction in the severity of disease or reduction in viral load in a patient infected with COVID-19.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in severity of disease in a subject infected with COVID-19, about 3, 4, 5 or 6 days after first dose of peroral administration of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in severity of disease in a subject infected with COVID-19, about 3 days after first dose of peroral administration of 400 μg/kg of body weight of daily dose of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in severity of disease in a subject infected with COVID-19, about 4 days after first dose of peroral administration of 400 μg/kg of body weight of daily dose of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in severity of disease a subject infected with COVID-19, about 5 days after first dose of peroral administration of 400 μg/kg of body weight of daily dose of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in severity of disease in a subject infected with COVID-19, about 6 days after first dose of peroral administration of 400 μg/kg of body weight of daily dose of the substance compared to placebo.

As used herein the term “severity of disease” refers to WHO 9-category ordinal scale, developed to have a standardised measure of the COVID-19 disease severity. Severity of disease is defined as a decrease on WHO ordinal scale score by at least one point.

It was surprisingly observed based on clinical trials data that a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, was able to lead to a statistically significant reduction in viral load in a subject infected with COVID-19.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in viral load in a subject infected with COVID-19, about 3, 4, 5 or 6 days after first dose of peroral administration of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in viral load a subject infected with COVID-19, about 3 days after first dose of peroral administration of 400 μg/kg of body weight of daily dose of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in viral load in a subject infected with COVID-19, about 4 days after first dose of peroral administration of 400 μg/kg of body weight of daily dose of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in viral load in a subject infected with COVID-19, at least 5 days after first dose of peroral administration of 400 μg/kg of body weight of daily dose of the substance compared to placebo.

In some embodiments, a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, is able to lead to a statistically significant reduction in viral load in a subject infected with COVID-19, about 6 days after first dose of peroral administration of 400 μg/kg of body weight of daily dose of the substance compared to placebo.

As used herein the term “viral load” refers to WHO 9-category ordinal scale, developed to have a standardised measure of the COVID-19 infectivity, transmission risk, disease phenotype, morbidity, mortality and disease severity relative to viral load. Viral load may be determined from samples obtained in the upper respiratory tract. Viral load can be determined using methods which would be known to those of skill in the art and these methods include but are not limited to colony forming unit (cfu) determination following cell culture, PCR. RT-PCT etc. Without wishing to be bound by theory, viral load may provide a better understanding of why transmission is observed in some instances, but not in others, especially among household contacts. Also, without wishing to be bound by theory, viral load in COVID-19 might correlate with infectivity, disease phenotype, morbidity and mortality.

In some embodiments, the reduction in viral load is at least 5%, 10%, 15%, 20%, 25%, 30%, 35%, 40%, 45%, 50%, 55%, 60%, 65%, 70%, 75%, 80%, 85%, 90%, 95% or 100% compared to control or placebo. In some embodiments the reduction in viral load is at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to control or placebo.

### Disease or Condition State and Biomarkers

Without wishing to be bound by theory, raised or elevated blood levels of different markers such as inflammatory markers. CBC markers and predictive markers have been associated with disease state such as for example viral infection. Thus, in some embodiments of the present invention, the predictive markers are selected from the group consisting of lymphocyte count; C reactive protein; D-dimer; LDH; ALAT; and ASAT. In some embodiments, the predictive marker is selected from C reactive protein and D-dimer. In some embodiments, the predictive marker is D-dimer. Conversely, it has been recognised in the art that reduction in the levels of these markers can be linked to improvement of disease state or absence of disease or disorder such as a caused by or associated with Coronaviridae infection such as MERS, SARS or COVID-19 infection.

Elevated levels of D-dimer may be defined as any D-dimer levels that are significantly above those D-dimer levels found circulating in the blood of healthy individuals. For example, D-dimer is present in the order from about 100 ng/mL to about 200 ng/mL in the majority of healthy or uninfected individuals, although such healthy or uninfected individuals may have D-dimer levels up to about 300 ng/mL or about 400 ng/mL without any underlying disease or active infection, or as low as 25 ng/mL, e.g. about 25 ng/mL to about 400 ng/mL and any intervening ranges, below 400 ng/mL, below 350 ng/mL, below 300 ng/mL, below 250 ng/mL, below 200 ng/mL below 150 ng/mL, below 100 ng/mL, and below 50 ng/mL, and any intervening ranges. Therefore. D-dimer levels in amounts above 400 ng/mL, especially those levels above 800 or 850 ng/mL, may be considered elevated, and thus may serve as the basis for assessing severity of disease or disorder caused by or associated with Coronaviridae infection such as MERS, SARS or COVID-19 infection. As described herein, D-dimer levels from about 400 ng/mL to about 5000 ng/mL along with any intervening ranges may be considered elevated and thus indicative of disease or disorder caused by or associated with Coronaviridae infection such as MERS, SARS or COVID-19 infection. The D-dimer level may be, for example, above 400 ng/mL, above 450 ng/mL, above 500 ng/mL, above 550 ng/mL, above 600 ng/mL, above 650 ng/mL, above 700 ng/mL, above 750 ng/mL, above 800 ng/mL, above 850 ng/mL, above 900 ng/mL, above 950 ng/mL, above 1000 ng/mL, above 1050 ng/mL, above 1100 ng/mL, above 1150 ng/mL, above 1200 ng/mL, above 1250 ng/mL, above 1300 ng/mL, above 1350 ng/mL, above 1400 ng/mL, above 1450 ng/mL, above 1500 ng/mL, above 1600 ng/mL, above 1700 ng/mL, above 1800 ng/mL, above 1900 ng/mL, above 2000 ng/mL, above 2500 ng/mL, above 3000 ng/mL, above 3500 ng/mL, above 4000 ng/mL, above 4500 ng/mL, above 5000 ng/mL, and any intervening ranges. The prediction of disease or disorder caused by or associated with Coronaviridae infection such as MERS, SARS or COVID-19 infection may be made with or without additional markers, i.e. elevated D-dimer levels may be considered a major predictive marker.

As a major predictive marker, elevated D-dimer levels may be used as reliable basis for determining severity and disease or disorder caused by or associated with Coronaviridae infection such as MERS, SARS or COVID-19 infection.

Consequently, as a major predictive marker, reduction in elevated D-dimer levels may be used as reliable basis for determining outcome of treatment of disease or disorder caused by or associated with Coronaviridae infection such as MERS, SARS or COVID-19 infection. Also, as a major predictive marker, any reduction in D-dimer levels can be used to show efficacy of treatment.

Thus, in some embodiments, the present treatment leads to reduction of one or more markers selected from the group of markers consisting of inflammatory markers, CBC markers and predictive markers, in a subject suffering from a disease or disorder caused by or associated with Coronaviridae infection such as MERS, SARS or COVID-19 infection.

In some embodiments, the present treatment leads to reduction of one or more predictive markers in a subject suffering from a disease or disorder caused by or associated with Coronaviridae infection such as MERS, SARS or COVID-19 infection.

In some embodiments, the present treatment leads to reduction of one or more predictive markers selected from the group predictive markers consisting of lymphocyte count; C reactive protein; D-dimer; LDH; ALAT; and ASAT, in a subject suffering from a disease or disorder caused by or associated with Coronaviridae infection such as MERS, SARS or COVID-19 infection.

In some embodiments, the present treatment leads to reduction of D-dimer marker levels in a subject suffering from a disease or disorder caused by or associated with Coronaviridae infection such as MERS, SARS or COVID-19 infection.

In some embodiments, the present treatment leads to reduction of D-dimer marker levels in a subject infected with COVID-19.

In some embodiments, the reduction of D-dimer is at least 5%, 10%, 15%, 20%, 25%, 30%, 35%, 40%, 45%, 50%, 55%, 60%, 65%, 70%, 75%, 80%, 85%, 90%, 95% or 100% or greater compared to control or placebo. In some embodiments the reduction of D-dimer is at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to control or placebo.

In some embodiments of the present invention, the reduction of the predictive marker in a treated subject is statistically significant compared to a placebo. In some embodiments of the present invention, the reduction of D-dimer marker levels in a subject infected with COVID-19 is statistically significant.

### Combination Treatment

Avermectin class of substances such as one or more of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof, can be administered in the present methods, uses or compositions in combination with (e.g. in a combined formulation with) other therapeutic agents for example agents that are useful as, for example, antiviral agents, anti-inflammatory agents, anti-neutrophil agents, anti-macrophage agents, NSAID, vaccines against COVID-19 or the like. Example vaccines include for example: BNT162b2 (mRNA based and developed by Pfizer-BioNTech—requiring two doses three weeks apart), mRNA-1273 (mRNA based—requiring 2 doses at 4 weeks interval—Moderna), Sputnik V (Russian Federation Health Authority—formerly Gam-Covid-Vac—2 doses at 3 weeks interval) etc.

In some embodiments, ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof, is used in combination with a vaccine against COVID-19. In some embodiments, ivermectin is used in combination with BNT162b2 vaccine. In some embodiments, ivermectin is used in combination with mRNA-1273 vaccine. In some embodiments, ivermectin is used in combination with Sputnik V vaccine.

Unless otherwise stated or apparent from the context (e.g. when discussed in reference to a specific formulation or administration regimen), references to the dose of one or more of Avermectins, derivatives and/or salts thereof, according to the invention (e.g. the dose of ivermectin derivatives and/or salts thereof) will be understood to refer to the dose of ivermectin (i.e. the dose of ivermectin itself, or the effective (i.e. equivalent) dose of ivermectin when administered in the form that includes or consists of one or more free form or in the form of a physiologically acceptable derivative and/or salt thereof.

In order that the invention may be readily understood and put into practical effect, particular embodiments will now be described by way of the following non-limiting examples.

### EXAMPLES

### Example 1

Treatment Methods and Protocols

Main clinical symptoms of COVID-19 infection have been reported and these include for example fever, cough, myalgia or fatigue, expectoration, sore throat, conjunctivitis and dyspnea plus a number of less manifested and recently reported clinical symptoms of almost all body systems and organs, including but limited to encephalitis, Meniere-like syndrome, gastrointestinal, cardio-vascular, renal and skin manifestations correlating with changes in a number of paraclinical and laboratory parameters. While most patients do not experience severe symptoms, and some remain asymptomatic or oligosymptomatic, one meta-analysis found that approximately 14-18% of cases were representing a severe clinical picture. Fatality rates are estimated to be approximately 4-7% (Crump A, 2017; Gloeckner et al., 2010).

A 3-stage classification of the disease was proposed by Siddigi and Mehra, 2020. The initial stage occurs at the time of inoculation and early establishment of the disease. For most people, this involves an incubation period associated with mild and often non-specific symptoms such as malaise, fever, sore throat and a dry cough. Treatment at this stage is primarily targeted towards symptomatic relief. Should a viable anti-viral therapy be proven beneficial, the target pool of infected patients during this stage may reduce duration of symptoms, minimize contagiousness and prevent progression of severity. The latter assumption may prove to be a powerful tool and a cornerstone in limitation of COVID-19 life-threatening and incapacitating possibilities.

In the second stage of established pulmonary disease, viral multiplication and localized inflammation in the lung is the norm. During this stage, patients develop an interstitial lung disease (viral pneumonia), with cough, fever and possibly hypoxia. Treatment would primarily consist of supportive measures and available more aggressive (and expensive) anti-viral therapies which are mainly administered via intravenous route of application. If hypoxia is not reverted via these additional treatments (Stage IIb), it is likely that patients will progress to requiring mechanical ventilation and, in that situation, the use of anti-inflammatory therapy may be useful.

A minority of COVID-19 patients will undergo transition into the third and most severe stage of illness, which manifests as an extra-pulmonary systemic hyperinflammation syndrome. In this stage, markers of systemic inflammation appear to be elevated, including cytokines and interleukin (IL). In this stage, shock, vasoplegia, respiratory failure and even cardiopulmonary collapse are discernible. Systemic organ involvement, even myocarditis, would manifest during this stage. Tailored therapy in Stage III hinges on the use of immunomodulatory agents to reduce systemic inflammation before it overwhelmingly results in multi-organ dysfunction.

Currently, our understanding of the clinical spectrum of 2019-nCoV infection is still limited. Complications such as severe pneumonia, respiratory failure, acute respiratory distress syndrome (ARDS), and cardiac injury, including fatal outcomes, have been reported on global scale. However, it is important to note that majority of reported cases were identified on the basis of their pneumonia diagnosis and thus may bias reporting toward more severe outcomes, so both treatment and description of the medical condition and progression of COVID19 represent an unmet challenge.

Pre-Clinical Studies

Acute oral studies with ivermectin in mice, rats, and monkeys have shown clear species and strain differences in sensitivity, with rodents being relatively sensitive to the CNS toxicity produced by the compound. Doses of 200 μg/kg in mice and slightly higher doses in rats have produced tremors and ataxia, while a 200 μg/kg dose was generally well tolerated in studies in a variety of species. Acute oral studies in rhesus monkeys demonstrated that the minimum toxic dose was 2,000 μg/kg or approximately 10-fold the clinical dose. Doses up to 24 mg/kg in this species (120-fold the clinical dose) produced only slight increases in the observed toxic effects including emesis, mydriasis, and sedation.

A repeated dose study of 2 weeks' duration in rhesus monkeys produced no adverse effects at doses up to 1,200 μg/kg/day. Fourteen-week studies in rats and dogs indicated no adverse effects at doses of 400 and 500 μg/kg/day, respectively.

Breeding performance in various species has not been adversely affected by ivermectin.

Ivermectin caused cleft palates in mice and rats at oral doses of 0.4 and 10 mg/kg/day respectively, and cleft palates and clubbed feet in rabbits dosed at 3 mg/kg/day.

Long-term studies have not been performed to evaluate the carcinogenic potential of ivermectin.

Ivermectin is negative in the Ames microbiological mutation and the mouse lymphoma mutation assays; it did not induce unscheduled DNA synthesis in human fibroblast cell culture, and thus, did not show any genotoxicity.

Significant lethality was observed in mice and rats after single oral doses of 25 to 50 mg/kg and 40 to 50 mg/kg, respectively. No significant lethality was observed in dogs after single oral doses of up to 10 mg/kg. At these doses, the treatment related signs that were observed in these animals include ataxia, bradypnea, tremors, ptosis, decreased activity, emesis, and mydriasis.

Clinical Studies

Pharmacokinetics

Following oral administration of ivermectin, plasma concentrations are approximately proportional to the dose. In two studies, after single 12-mg doses of ivermectin in fasting healthy volunteers (representing a mean dose of 165 mcg/kg), the mean peak plasma concentrations of the major component (H2B1a) were 46.6 (±21.9) (range: 16.4-101.1) and 30.6 (±15.6) (range: 13.9-68.4) ng/mL, respectively, at approximately 4 hours after dosing. Ivermectin is metabolized in the liver, and ivermectin and/or its metabolites are excreted almost exclusively in the feces over an estimated 12 days, with less than 1% of the administered dose excreted in the urine. The plasma half-life of ivermectin in man is approximately 18 hours following oral administration.

The safety and pharmacokinetic properties of ivermectin were further assessed in a multiple-dose clinical pharmacokinetic study involving healthy volunteers. Subjects received oral doses of 30 to 120 mg (333 to 2000 mcg/kg) ivermectin in a fasted state or 30 mg (333 to 600 mcg/kg) ivermectin following a standard high-fat (48.6 g of fat) meal. Administration of 30 mg ivermectin following a high-fat meal resulted in an approximate 2.5-fold increase in bioavailability relative to administration of 30 mg ivermectin in the fasted state.

In vitro studies using human liver microsomes and recombinant CYP450 enzymes have shown that ivermectin is primarily metabolized by CYP3A4. Depending on the in vitro method used, CYP2D6 and CYP2E were also shown to be involved in the metabolism of ivermectin but to a significantly lower extent compared to CYP3A4. The findings of in vitro studies using human liver microsomes suggest that clinically relevant concentrations of ivermectin do not significantly inhibit the metabolizing activities of CYP3A4, CYP2D6, CYP2C9, CYP1A2, and CYP2E1.

General Safety Considerations

Until now ivermectin is used mainly in people suffering from parasitic diseases such as filarial diseases and other worm-related infections and diseases. In these cases, it is considered that most of the adverse events are caused not by the drug itself, but rather from the death of parasites, killed by the drug.

Common side effects, observed during treatment with ivermectin are headache, dizziness, drowsiness, muscle pain, abdominal pain, nausea, vomiting or diarrhea, pruritus, fatigue, hypotension.

For patients, treated with ivermectin for parasitic worm infections the following side effects were also observed—joint or muscle pain, tender/swollen lymph nodes, eye swelling/redness/pain, weakness, vision changes, itching, rash, and fever, loss of appetite, shaking or trembling, sleepiness.

Selection, Study Objectives and Purpose

Unless indicated otherwise, “study population” or “S-population” is used interchangeably with the term “trial population” or “T-population” and refer to subjects that fulfill all eligibility criteria to be involved in the study to whom the study substance such as ivermectin is administered. The S-population may comprise randomised subjects to whom the study substance of the present invention is administered. Not all S-population subjects would be administered with ivermectin e.g. control subjects, to whom placebo is administered.

Selection of Study Population

The subjects must fulfill all eligibility criteria to be involved in this study. No exemptions from any in-/exclusion criteria will be allowed. If any deviation from eligibility is retrospectively detected for an already randomised subject, the investigator and sponsor must decide immediately whether it is safe to treat this subject further within the study.

Inclusion Criteria
1. Male or female patients at least 18 years and up to 75 years old
2. Signed informed consent
3. Admitted to hospital for treatment of COVID-19.
4. Patient within 7 days from symptom onset and within 72 hours after laboratory diagnosis (SARS-CoV-2 RT-PCR)
5. Mild to Moderate COVID-19 disease defined as clinical status category up to 3 on the WHO 9-point ordinal scale, and/or NEWS2 score of 0 to 4 inclusive.
   Hospitalized, no oxygen treatment
6. Presence of at least 1 symptom characteristic for COVID-19 disease, e.g. fever, cough, sore throat, myalgia, fatigue, GI disorders, skin lesions, anosmia, ageusia, headache, etc
7. In women of childbearing potential, negative pregnancy test and commitment to use contraceptive method throughout the study.

Screening Period and Randomization/Day 1

Patients are screened for 24-36 hours. The patients are randomized on Day 1 and treated with ivermectin/placebo+SoC (if required) for 3 days (Day 1-Day 3). It is encouraged to screen potential participants immediately at the day of hospitalization (including informed consent, assessment of inclusion/exclusion criteria, screening laboratory tests all done by the local central lab, assessment of clinical symptoms criteria) and randomize patients on the same day (thus, Screening period and Day 1 will coincide). To assess eligibility criteria, central local laboratory values obtained within 24-36 hours of randomization will be used, except for pre-study testing of positive status of COVID-19 infection where a 72-hours window.

Primary Objective

The primary objective of this study is to evaluate the efficacy of ivermectin plus investigator's choice of standard of care therapy (SoC) vs placebo plus SoC in the treatment of coronavirus disease 2019 (COVID-19) based on the qualitative virological clearance i.e. remission, in upper respiratory tract swab samples on Day 7.

Day 7 is an empirically driven timepoint based on the current literature data to serve as a critical milestone in the disease progression.

However, in vast majority of cases, this would correspond to [days 10-12] of the subject illness development and at this timepoint a possible conversion of RT-PCR test occurs from positive to negative again in the majority of the cases. This conversion of RT-PCR test is supposed to correlate with a successful healing and recovery process such as partial or complete remission, for the particular subject.

Secondary Objectives Include

1. Evaluate the efficacy of Ivermectin plus investigator's choice of standard of care therapy (SoC) vs placebo plus SoC in the treatment of coronavirus disease 2019 (COVID-19) based on clinical improvement on Day 7 and Day 14, and

2. Evaluate the efficacy of Ivermectin plus investigator's choice of standard of care therapy (SoC) vs placebo plus SoC in the treatment of coronavirus disease 2019 (COVID-19) based on clinical recovery on Day 7 and Day 14.

Additional Secondary Objectives Include:

1. To evaluate the efficacy of Ivermectin plus investigator's choice of standard of care therapy (SoC) vs placebo plus SoC in the treatment of coronavirus disease 2019 (COVID-19) based on different additional clinical endpoints; and

2. To evaluate the effect of treatment with Ivermectin plus investigator's choice of standard of care therapy (SoC) vs placebo plus SoC on hematology and inflammatory markers

Safety Objective

To evaluate the safety of Ivermectin plus investigator's choice of standard of care therapy (SoC) vs placebo plus SoC in the treatment of coronavirus disease 2019 (COVID-19) based on adverse events, vital signs, laboratory parameters, ECG.

Exploratory Objective

To trace SARS-CoV-2 viral load dynamics in upper respiratory tract frozen samples by quantitative RT-PCR until Day 14.

The time points for this objective will be Days 3 to 7, 9, 12 and 14.

Primary and Secondary Endpoints

Primary endpoint based on which the primary study objective will be assessed is proportion of patients who have shown negative SARS-CoV-2 PCR test on Day 7.

The secondary endpoints, related to the secondary study objectives are the following:
a. Number of patients achieving clinical improvement on Day 7
b. Number of patients achieving clinical improvement on Day 14
c. Number of patients achieving clinical recovery on Day 7
d. Number of patients achieving clinical recovery on Day 14

Endpoints, related to the additional secondary objectives are:
Time to conversion to a negative SARS-CoV-2/COVID-19 (qualitative) test until Day 28
Time to achieving clinical improvement until Day 28
Time to achieving clinical recovery until Day 28
Rate of subjects converted to negative SARS-CoV-2/COVID-19 (qualitative) test on Day 4
Rate of subjects converted to negative SARS-CoV-2/COVID-19 (qualitative) test on Day 9 and Day 12
Rate of subjects converted to negative SARS-CoV-2/COVID-19 (qualitative) test on Day 14
Time to hospital discharge
Number of patients who have needed high flow oxygen therapy
Number of patients who have needed ICU treatment
Inflammatory, CBC and predictive markers include: Lymphocyte count; C reactive protein; D-dimer; LDH; ALAT; and ASAT.
   The parameters used for the assessment of the Safety objective are:
Adverse events
Laboratory parameters
ECG
Vital signs

Description of the Treatment

Test Product

Ivermectin, 3 mg tablets, packed in aluminum/PVC foil, 4 tablets per blister.

Formulation: Tablets with 3 mg ivermectin.

Administration: Certain number of tablets, corresponding to 400 μg/kg body weight of ivermectin should be administered with water in a fast state—two (2) hours after and before meal, if applicable. Day 1 is defined as the day of the first IMP/placebo administration.

The first IMP on should be administered either in the evening of the day of the screening (thus, screening and Day 1 will coincide), or in the morning of Day 1 (the day right after completion of screening activities). IMP administration in the next two days (Days 2 and 3) should be done approximately in the same time as the one of Day 1 (either in the evening or in the morning).

Placebo

Matching placebo, once-daily oral administration as described for the test product, identical number of tablets as given for Ivermectin.

Standard-of-Care (SoC)

During the 3 days of IMP/placebo administration the standard medications that investigators would customarily use to treat mild to moderate COVID-19 symptoms in their clinical practice are allowed. This is limited to antipyretic, anti-inflammatory, antitussive or else symptomatic treatments drugs plus infusions and/or O2 saturation in case necessary. During the 3 days of study drug administration and until Day 7 medications with approved antiviral indication, antibiotics, as well as Choroquine and Hydroxychloroquine should be avoided, if the condition of the patient allows this.

After Day 7 the investigator's choice of SoC in this study can include supportive pharmaceutical treatments (including medications that are approved in other indications but that the investigator customarily uses in COVID-19 patients), medications with any approved antiviral indication, intravenous fluids, supplemental oxygen, noninvasive and invasive ventilation, antibiotic agents, vasopressor support, RRT, and ECMO.

Ivermectin Treatment

Excipients: Butylhydroxyanysole, anhydrous citric acid, pregelatinized starch, cellulose microcrystalline, magnesium stearate:
Formulation: Tablet containing 3 mg ivermectin
Matrix: White uncoated tablets, diameter of 6 mm and weight 60 mg
Dose strength: Total daily dose of 400 μg/kg body weight.

Dose Selection, Treatment Dose and Administration

Certain number of tablets, corresponding to 400 μg/kg of ivermectin are administered with water in a fast state—two (2) hours after and before meal, if applicable.

Day 1 is defined as the day of the first IMP/placebo administration.

The first IMP on Day 1 should be administered either in the evening of the screening day (in this case the screening and Day 1 will coincide), or in the morning of Day 1 (the day right after completion of screening activities). IMP administration in the next two days (Days 2 and 3) should be done approximately in the same time as the one of Day 1 (either in the evening or in the morning).

In Table 1 below the examples of the number of tablets, that should be administered based on the body weight of the patient are presented.

TABLE 1
Dose/Tablet correlations

| | Daily dose in mg | Tablets |
|---|---|---|
| Body weight | (400 mcg/kg) | per day |
| 15-24 | 6 | 2 |
| 25-35 | 12 | 4 |
| 36-50 | 18 | 6 |
| 51-65 | 24 | 8 |
| 66-79 | 30 | 10 |
| 80-90 | 33 | 11 |
| 91-95 | 36 | 12 |
| 96-101 | 39 | 13 |
| ≥102* | 41 | 14 |

*Note:
Patients above 102 kg body weight are not given more than 14 tablets/41 mg daily dose.

Viral Clearance in Upper Respiratory Tract Swab Samples (Qualitative)—Remission

Upper respiratory tract swab samples will be collected on Days 3, 4, 5, 6, 7, 9, 12 and 14 or until two negative results are obtained. Between Day 15 and Day 28 RT-PCR shall be done upon the discretion of the Investigator until 2 consecutive negative tests.

No further RT-PCR tests will be done after 2 consecutive negative tests are obtained at any time point of the study. Swab samples should be obtained from the nasopharynx and oropharynx with two different swabs and combined in one tube, containing universal transport media. Samples are collected preferably in the mornings of each study day. Full instructions for collection, preparation, labelling, storage and shipment of samples are provided in the Laboratory Manual.

Clinical Disease Severity Assessment

The clinical disease severity during the study will be assessed based on the WHO Ordinal Scale for Clinical Status (Table 2). The WHO scale is a 9-category ordinal scale and was developed to have a standardized measure of the COVID-19 disease severity. The scale comprises patient status (outpatient or hospitalized), virus status (infected yes or no), limitation of activities, oxygen support, and organ support.

TABLE 2
WHO Ordinal Scale for Clinical Status

| Patient state | Descriptor | Score |
|---|---|---|
| Uninfected or no | No clinical or virological evidence of infection | 0 |
| longer infectious | | |
| Mild Disease | No limitation of activities* | 1 |
| | Limitation of activities* | 2 |
| Moderate Disease | No oxygen therapy* | 3 |
| | Oxygen by mask or nasal prongs* | 4 |
| Severe Disease | Non-invasive ventilation or high- flow oxygen* | 5 |
| | Intubation and mechanical ventilation* | 6 |
| | Hospitalized, virus-positive, ventilation | 7 |
| | plus additional organ support (pressors, | |
| | RRTT, ECMO)* | |
| Dead | Death* | 8 |

*All patients show positive samples for SARS-CoV-2/COVID-19

Statistical Design

O'Brien-Fleming method for normally distributed data (O'Brien, P. C. and Fleming, T. R. 1979) are used. At each stage, the current and future boundaries are calculated based on the accumulated information proportion. Conditional and predictive power for future stages will be also given.

A group sequential analysis consists of a series of stages where a decision to stop or continue is made at each stage.

First Interim Stage:

The study begins, and response data is collected for subjects, moving toward the first-stage target number of subjects, until a decision to perform an analysis on the existing data is made. The analysis at this point is called the first stage.

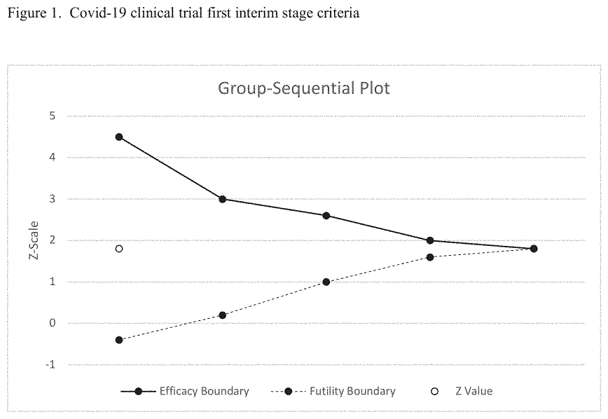

The first stage information is divided by the maximum information to obtain the stage one information proportion (or information fraction) FIG. 1. This information proportion is used in conjunction with the spending function(s) to determine the alpha and/or beta spent at that stage. In turn, stage one boundaries, corresponding to the information proportion, are calculated.

A z-statistic is calculated from the raw proportion difference. The stage one z-statistic is compared to each of the stage one boundaries. Typically, if one of the boundaries is crossed, the study is stopped (non-binding futility boundaries may be an exception).

If none of the boundaries are crossed the study continues to the next stage.

If none of the boundaries are crossed it may also be useful to examine the conditional power or stopping probabilities of future stages.

Conditional power and stopping probabilities are based on the user-specified supposed true difference.

Second Stage:

At the final stage, only the decision of efficacy or futility is made.

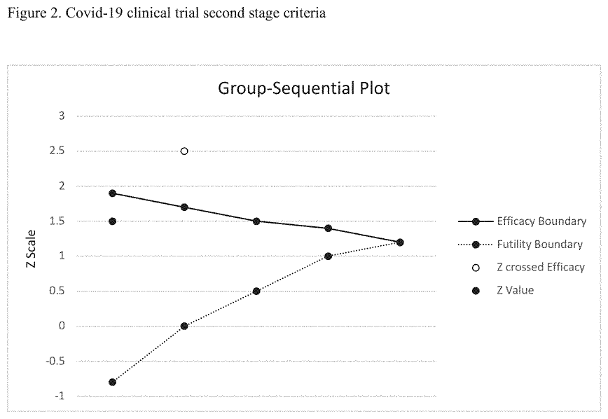

The final stage z-statistic is computed from the sample proportions of the complete data from each group —FIG. 2. The z-statistic is compared to the boundary and a decision of efficacy or futility is made.

Clinical Trials Data

A) Primary Endpoint Analysis

According to (Table 3), the results of this analysis demonstrate that the treatment arm which includes HUVE-19 has beneficial effect on the viral clearance immediately after the treatment with HUVE-19 (Day 4), proven with 2 consecutive negative PCR test results on this day.

On Day 4, 52% of the patients on Ivermectin have negative test results against 42% on Placebo arm (data not shown). Distribution of the two consecutive negative PCR tests in two consecutive days).

Conclusion may be done that PCR test may be used as an endpoint to compare two treatment arms. The effect of Ivermectin appears earlier than day 7 and PCR test may able to measure the true effect.

TABLE 3
Day of second negative PCR test
Analysis Variable: Day of second negative PCR test

| Treatment | N | Mean | Std Dev | Minimum | Maximum |
|---|---|---|---|---|---|
| Reference | 50 | 6.06 | 2.85 | 4.00 | 17.00 |
| Test | 50 | 5.52 | 2.79 | 3.00 | 17.00 |
| Difference | | 0.54 | 2.82 | | |
| p-value | | 0.3407 | | | |

B) Secondary Endpoints Analysis

HUVE-19 treatment arm demonstrated statistically significant difference on Days 4 (p=0.02 . . . ) compared to the placebo arm. The same picture was observed on Day 5, with difference 18% (p=0.03 . . . ), regarding the endpoint Clinical improvement. This endpoint was measured using WHO 9-category ordinal scale, developed to have a standardized measure of the COVID-19 disease severity. Clinical improvement is defined as a decrease on WHO ordinal scale score by at least one point. For our statistical calculations from data obtained from the clinical trials we used the method described above i.e. O'Brien-Fleming method for normally distributed data.

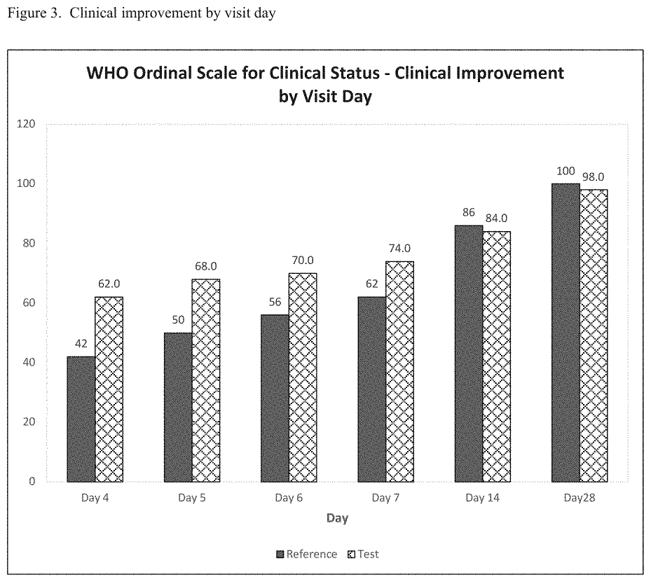

It was clear that Ivermectin gives better results at days 4 and 5, and after that the difference decreases and goes statistically insignificant (FIG. 3—Clinical improvement by visit day).

C) Odds Ratio Analysis of Clinical Improvement

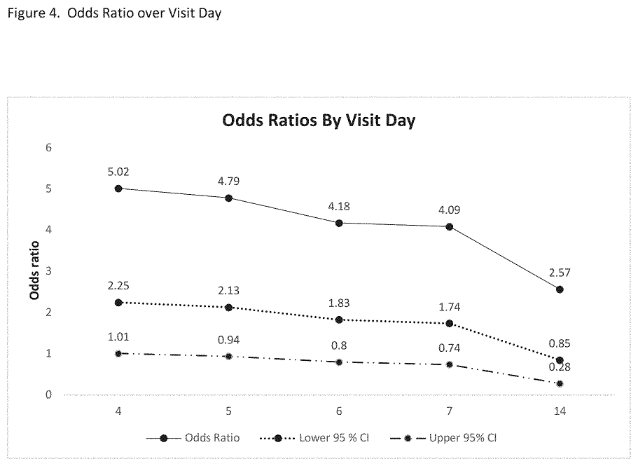

Odds ratio measurement over the visit day clearly demonstrated that the chances of clinical improvement for Ivermectin arm are more than twice higher than for SoC arm only at the fourth day (FIG. 4 Odds ratio analysis).

At the fifth and the next days the odds ratio values decrease but remain higher for Ivermectin.

D) Efficacy Conclusion:

Ivermectin treatment arm compared to the Placebo treatment arm definitely shows clinical benefit in the early days of the infection such as day 4 and day 5 after initial dose and especially in the parameter Clinical improvement.

Endpoints, Related to the Additional Secondary Objectives

Time to conversion to negative PCR test until Day 28 endpoint showed that the difference is 0.54 days, and is statistically insignificant (p=0.3407).

Time to hospital discharge endpoint showed that there is no statistically significant difference (p=0.2755) in time to discharge from hospital for two arms.

Inflammatory, CBC and predictive markers—Lymphocyte count
C reactive protein
D-dimer
LDH
ALAT/ASAT

The review of inflammatory parameters and predictive markers (CRP and D-dimer plus hepatic enzymes panel) as well as the lymphocyte count taken as a separate predictive sign, has shown that while there are no major differences in the baseline values of Lymphocyte count parameter plus the hepatic enzymes panel (ASAT/ALAT and LDH) and their respective dynamic changes for each of them.

However, the clinical data for the CRP and D-dimer parameters is unexpectedly different with potential clinical relevance as described below:

CRP Parameters

CRP values baseline values and their dynamic show the following—the randomized assignment of study subjects at baseline shows uneven distribution of subjects with higher levels of CRP value for the test treatment arm compared with the referent one (mean values of 64.43 mg/l for Test arm vs 41.15 mg/l for Referent arm; resp median values are 39.44 mg/l for Test arm vs 28.82 mg/l for Referent arm) which may lead to the conclusion that patients enrolled in the Test Treatment arm are represented with laboratory signs of more severe inflammatory process as compared to the ones enrolled in the Referent arm.

The latter observation corresponds to comparison in general medical condition of subjects' treatment arms as measured by WHO Grade level.

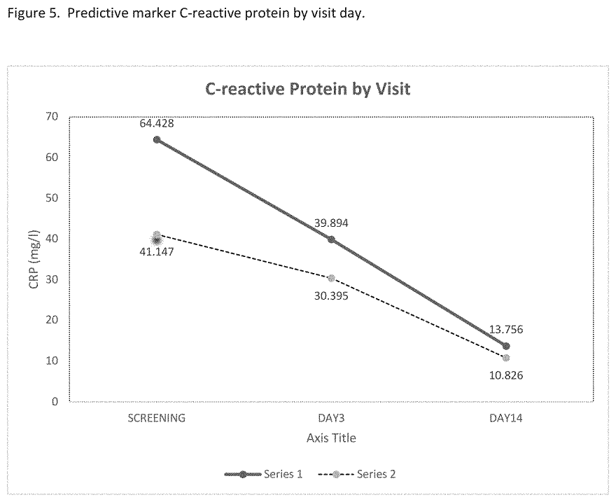

Furthermore, changes in CRP values in course of time show that both arms (Test and Referent) are diminishing values reaching a relative parity at Day 14 (mean values of 13.76 mg/l (Test arm) vs 10.83 mg/l (Referent arm); respective median values of 3.70 mg/l vs 3.03 mg/l (FIG. 5. C reactive protein by visit).

D-Dimer Parameter Show the Following Surprising Clinically Relevant Observations:

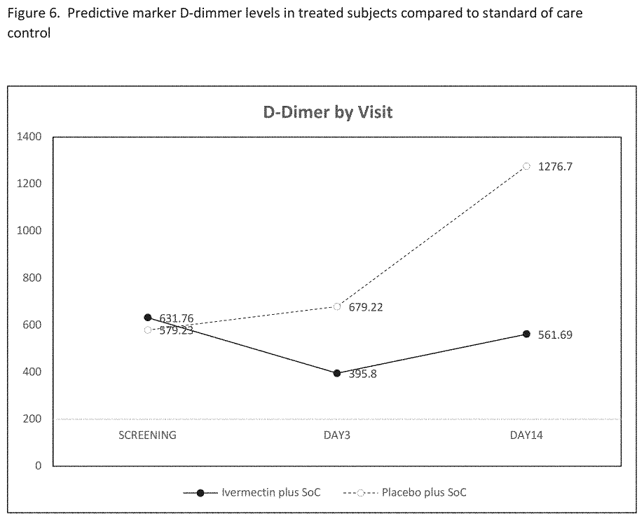

While at the values of subjects for this parameter show similar values registered for the subjects in both treatment arms (mean 631.76 ng/ml for ivermectin treatment arm vs 579.23 ng/ml for placebo arm respectively) further observations show respective significant changes in the course of study: the mean values for Test/Referent arms on Day 3 are 395.8 ng/ml vs 679.22 ng/ml and on Day 14 the trend in differences keeps consistency in the treatment arms with values for Test/Referent arms as follows (561.69 ng/ml vs 1276.7 ng/ml)(FIG. 6. Reduction in D-dimer levels per visit relative to standard of case control (SoC)).

Some respective medical conclusions may be drawn from the above review of lab parameters in combination with the medical assessment results but they are not definite due to the relatively small sample size and the missing values at Day 7 and Day 28.

Treatment Efficacy Conclusion:

General conclusion regarding the efficacy of HUVE-19 ivermectin treatment arm compared to the Placebo treatment arm is that the tested product definitely shows clinical benefit in the early days of the infection, especially in the parameter Clinical improvement. The latter is demonstrated by the WHO Score measurement and also by the evolution of some inflammatory parameters and predictive markers (CRP and D-dimer).

The study clearly demonstrated that the PCR test results are not very adequate endpoint to compare two treatment arms, albeit the treatment arm does have beneficial effect on the viral clearance immediately after the treatment proven with 2 consecutive negative PCR test results on Day 4, where 52% of the patients on Ivermectin have negative test results against 42% on Placebo arm. These clinically relevant findings were surprising and unexpected to the inventors as well as the medial practitioners involved in the trials.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognised that various modifications are possible within the scope of the disclosure claimed. It will also be appreciated that the method(s), use(s), compositions(s) and/or administrations(s) may be subject to numerous rearrangements, modifications and substitutions without departing from the scope of the present disclosure as set forth and defined by the following claims.

### REFERENCES

Sun P, Qic S, Liu Z, Ren J, Li K, Xi J. Clinical characteristics of hospitalized patients with SARS-CoV-2 infection: A single arm meta-analysis. J Med Virol. 2020; March 17. Doi: 10.1002/jmv.25735.
Crump A. Ivermectin: enigmatic multifaceted ‘wonder’ drug continues to surprise and exceed expectations. J Antibiot (Tokyo) 2017: 70: 495-505.
Siddiqi H K, Mehra M R. COVID-19 Illness in Native and Immunosuppressed States: A Clinical-Therapeutic Staging Proposal. Journal of Heart and Lung Transplantation 2020 https://www.ihltonline.org/article/S1053-2498(20)31473-X/fulltext doi: 10.1016/j.healun.2020.03.012.
Gloeckner C, Gamer A L, Mersha F. Oksov Y, Tricoche N, Eubanks L M, Lustigman S, Kaufnann G F, Janda K D. Repositioning of an existing drug for the neglected tropical disease Onchocerciasis. Proc Natl Acad Sci USA 2010; 107: 3424-9.
World Health Organization (2020): WHO Director-General's opening remarks at the media briefing on COVID-19-11 Mar. 2020 https://www.who.int/dL/speechesfdetail/who-director-general-s-opening-remarks-at-the-media-briefing-on-covid-19-11-march-2020
O'Brien. P. C. and Fleming, T. R. 1979. A multiple testing procedure for clinical trials. Biometrics, 35, 549-556.

1-57. (canceled)

58. A substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof, for use in the treatment of a subject infected with a Coronaviridae family member Such as MERS, SARS or COVID-19, wherein the subject is administered with a therapeutically effective amount of the substance in a free form or in the form of a physiologically acceptable derivative and/or salt thereof, and wherein the therapeutically effective amount is achieved by a regimen of administration of the substance in a free form or in the form of a physiologically acceptable derivative and/or salt thereof.

59. A substance according to claim 58, wherein the regimen of administration of the substance in a free form or in the form of a physiologically acceptable derivative and/or salt thereof comprises one or more administrations selected from peroral, pulmonary, intravenous, intramuscular and subcutaneous.

60. A substance according to claim 58, wherein the substance leads to a reduction of one or more markers selected from the group consisting of inflammatory markers, CBC markers and predictive markers, in a subject infected with COVID-19, and wherein the one or more predictive marker is selected from the group consisting of lymphocyte count; C reactive protein; D-dimer; LDH; ALAT; and ASAT, and wherein the reduction of the one or more predictive marker in a treated subject is statistically significant compared to a placebo.

61. A substance according to claim 60, wherein the reduction of D-dimer is at least 5%, 10%, 15%, 20%, 25%, 30%, 35%, 40%, 45%, 50%, 55%, 60%, 65%, 70%, 75%, 80%, 85%, 90%, 95% or 100% or greater compared to placebo, or wherein the reduction of D-dimer is at least 1.5, 2, 2.5, 3, 3.5, 4, 4.5, 5, 5.5, 6, 6.5, 7, 7.5, 8, 8.5, 9, 9.5, or 10-fold or greater compared to placebo.

62. A substance according to claim 58, wherein the substance leads to a statistically significant clinical improvement in a subject infected with MERS, SARS or COVID-19 about 3, 4, 5 or 6 days after first dose of peroral administration of the substance compared to placebo.

63. A substance according to claim 58, wherein the substance leads to a statistically significant reduction in severity of disease in a subject infected with MERS, SARS or COVID-19 about 3, 4, 5 or 6 days after first dose of peroral administration of the substance compared to placebo.

64. A substance according to claim 58, wherein the substance leads to a statistically significant reduction of viral load in a subject infected with MERS, SARS or COVID-19 about 3, 4, 5 or 6 days after first dose of peroral administration of the substance compared to placebo.

65. A method of therapeutic treatment of a subject infected with a Coronaviridea family member such as MERS, SARS or COVID-19 with a substance selected from the group consisting of ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, in a free form or in the form of a physiologically acceptable derivative and/or salt thereof.

66. A method of therapeutic treatment of a subject according to claim 65, wherein the substance is ivermectin in a free form or in the form of a physiologically acceptable derivative and/or salt thereof, wherein the substance is in a form suitable for administering a dose of the substance in a dose or as divided doses or sub-doses administered at appropriate intervals per day.

67. A method of therapeutic treatment of a subject according to claim 65, wherein the substance in administered as one, two, three, four or more doses or sub-doses per day or per administration and wherein the dose of substance per administration will typically be in the range of about 0.1 to 2000 mg/kg of body weight, about 0.15 to 1750 mg/kg of body weight, about 0.2 to 1700 mg/kg of body weight, about 0.3 to 1500 mg/kg of body weight, about 0.5 to 1250 mg/kg of body weight, about 1 to 1000 mg/kg of body weight, about 2 to 900 mg/kg of body weight, about 3 to 800 mg/kg of body weight, about 4 to 700 mg/kg of body weight, about 5 to 600 mg/kg of body weight, about 10 to 500 mg/kg of body weight, administered as one, two, three, four or more doses or sub-doses per day or per administration.

68. A method of therapeutic treatment of a subject according to claim 65, wherein the substance is adapted for peroral administration and wherein for peroral administration, a daily dose will typically be within the range of about 10 to 1500 μg/kg of body weight, about 20 to 1250 μg/kg of body weight, about 30 to 1000 μg/kg of body weight, about 500 to 750 μg/kg of body weight.

69. A method according to claim 68, wherein the peroral administration daily dose is 400 μg/kg of body weight.

70. A composition for treatment, of Coronaviridae family member infection, the composition comprising ivermectin, avermectin, doramectin, selamectin, moxidectin, emamectin, eprinomectin, milbemectin, abamectin, milbemycin oxime, nemadection and the macrolide derivatives thereof absent sugar residue attached at carbon 13, alone or in combination, in a free form or in the form of a physiologically acceptable derivative and/or salt thereof, wherein the Coronaviridea family member is selected from the group consisting of MERS, SARS or COVID-19.

71. A composition according to claim 70, wherein the treatment leads to amelioration, prophylaxis, or reversal of a disease or disorder, or of at least one discernible symptom thereof, caused by or associated with Coronaviridae family member infection such as MERS, SARS or COVID-19 infection.

72. A composition according to claim 70, wherein the treatment leads to amelioration, prophylaxis, or reversal of a disease or disorder, or of at least one discernible symptom thereof, caused by or associated with Coronaviridae infection such as MERS, SARS or COVID-19 infection, by at least 10%, at least 20%, at least 30%, at least 40%, at least 50%, at least 60%, at least 70%, at least 80%, at least 81%, 82%, 83%, 84%, 85%, 86%, 87%, 88%, 89%, 90%, 91%, 92%, 93%, 94%, 95%, 96%, 97%, 98%, 99% or 100%.
